(19) United States
(12) Patent Application Publication
(10) Pub. No.: US 2025/0110107 A1
(43) Pub. Date: Apr. 3, 2025

(54) SCRATCH ASSAY DEVICE

(71) Applicant: University of North Dakota, Grand Forks, ND (US)

(72) Inventors: Nicholas Bittner, Grand Forks, ND (US); Ghoulem Ifrene, Grand Forks, ND (US)

(21) Appl. No.: 18/905,500

(22) Filed: Oct. 3, 2024

Related U.S. Application Data
(60) Provisional application No. 63/542,200, filed on Oct. 3, 2023.

(51) Int. Cl. G01N 33/483

(52) U.S. Cl. CPC G01N 33/4833

(57) ABSTRACT

A device for studying cell migration, wound healing, and/or cell-cell interaction in a cylindrical well of a cell culture well plate includes a central body having opposing first and second sidewall surfaces and opposing first and second ends, wherein the first end is configured to engage a bottom of the well, a first wing extending outward from the first sidewall surface between the first and second ends, and a second extending outward from the second sidewall surface between the first and second ends. Each of the first wing and the second wing is biased outward to engage an inner wall of the well.

### CROSS-REFERENCE TO RELATED APPLICATION(S)

This application claims the benefit of U.S. Provisional Application No. 63/542,200 filed Oct. 3, 2023, for “SCRATCH ASSAY DEVICE” by G. Ifrene and N. Bittner.

### STATEMENT OF GOVERNMENT INTEREST

This invention was made with government support under OIA-1946202 awarded by the National Science Foundation. The government has certain rights in the invention.

### BACKGROUND

The present application is generally directed to cellular culturing and more particularly to a scratch assay device.

A scratch assay involves creating a “scratch” or a controlled wound in a cell monolayer using a fine tool. This technique is utilized to study cell migration, wound healing, and cell-cell interactions. An issue with current scratch assay technology is the lack of uniformity in creating scratches manually, which can lead to inconsistencies in the experimental results.

### SUMMARY

A device for studying cell migration, wound healing, and/or cell-cell interaction in a cylindrical well of a cell culture well plate includes a central body having opposing first and second sidewall surfaces and opposing first and second ends, wherein the first end is configured to engage a bottom of the well, a first wing extending outward from the first sidewall surface between the first and second ends, and a second extending outward from the second sidewall surface between the first and second ends. Each of the first wing and the second wing is biased outward to engage an inner wall of the well.

The present summary is provided only by way of example, and not limitation. Other aspects of the present disclosure will be appreciated in view of the entirety of the present disclosure, including the entire text, claims and accompanying figures.

### BRIEF DESCRIPTION OF THE DRAWINGS

FIG. 1 is a perspective view of a first end of a scratch assay device.

FIG. 2 is a perspective view of second end of the scratch assay device.

FIG. 3 is a plan view of the second end of the scratch assay device.

FIG. 4 is a plan view of the first end of the scratch assay device.

FIG. 5 is a perspective side view of the scratch assay device.

FIG. 6 is a perspective view of a first end of a scratch assay device according to another embodiment of the present disclosure.

FIG. 7 is a perspective view of a separable shoe for the scratch assay device of FIG.

FIG. 8 is a side view of the shoe.

FIG. 9 is a plan view of a first end of a scratch assay device according to yet another embodiment of the present disclosure.

FIG. 10 is a top view of a scratch assay device disposed in a well cell culturing plate.

While the above-identified figures set forth embodiments of the present invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale, and applications and embodiments of the present invention may include features, steps and/or components not specifically shown in the drawings.

### DETAILED DESCRIPTION

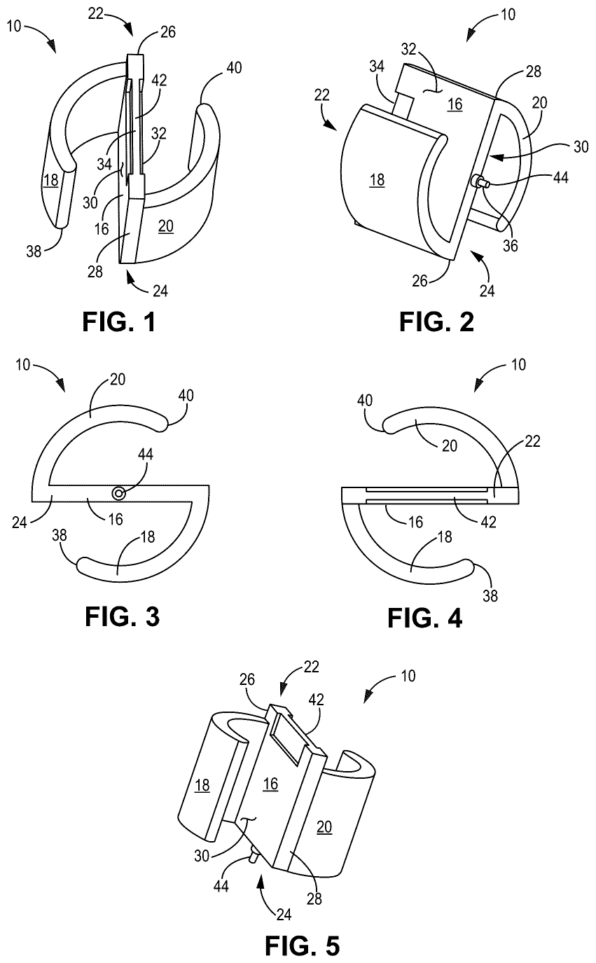

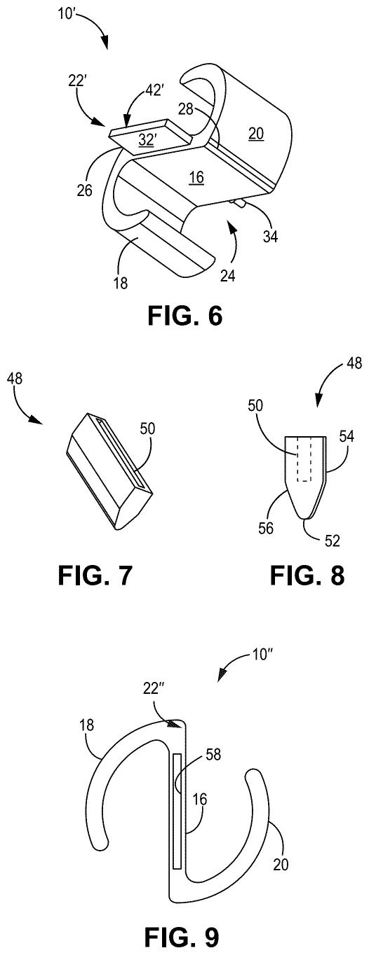

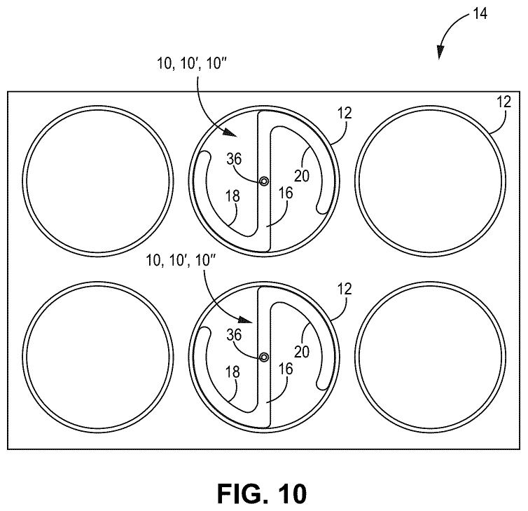

FIGS. 1-5 illustrate scratch assay device 10. FIG. 6 shows scratch assay device 10′, which is substantially similar to scratch assay device 10. FIGS. 7 and 8 show a shoe for use with scratch assay device 10′. FIG. 9 scratch assay device 10″, which is substantially similar to scratch assay device 10′. FIG. 10 illustrates one of scratch assay devices 10, 10′, and 10″ disposed in well 12 of well cell culturing plate 14. FIGS. 1-10 are discussed together herein.

The scratch assay devices of the present disclosure are configured to help standardize data collection for cellular culturing research. The disclosed scratch assay devices are specialized tools designed to fit into a well of a standard well culturing plate (e.g., well 12 of well plate 14 shown in FIG. 10). The disclosed scratch assay devices are equipped with a compliance mechanism that allows the scratch assay device to be securely held in place within the well. The purpose of the scratch assay device is to aid in performing a scratch (wound) assay, a widely used experimental technique in cell biology. A scratch assay involves creating a “scratch” or a controlled wound in a cell monolayer using a fine tool. This technique is utilized to study cell migration, wound healing, and cell-cell interactions. The disclosed scratch assay devices are configured to provide a standardized and consistent scratch size and shape. By providing a standardized and consistent scratch size and shape, the scratch assay devices ensure that the scratch assay is performed with precision and reproducibility. This allows researchers to compare and analyze cell migration and wound healing processes accurately, enabling a better understanding of cellular behaviors and mechanisms involved in various physiological and pathological conditions.

Each of the disclosed scratch assay devices is a versatile apparatus designed specifically for wound healing assays, providing researchers with a convenient and efficient tool for studying cell migration and invasion. The scratch assay devices are specifically designed to fit directly into a standard well (e.g., well 12 shown in FIG. 10) of a commercially available well cell culturing plate (e.g., well plate 14 shown in FIG. 10), allowing for seamless integration into existing laboratory workflows. The consistent size and shape of the “scratch” provided by the scratch assay device allows for a standardized method that enables improved or easier quantification of data especially when sourced from multiple laboratories. Although referencing a “scratch” assay, the disclosed scratch assay devices are not configured to scratch or remove cells from a cellular monolayer but are instead configured to be placed in a well prior to cell culturing. Cells can grow up to a portion of the scratch assay device in contact with a bottom surface of the well such that when the scratch assay device is removed from the well, a void in the shape of the portion of the scratch assay device to which cells grew up to is void of cells.

Referring now to FIGS. 1-5, scratch assay device 10 consists of two main components: central body 16 and two wings—first wing 18 and second wing 20. Central body 16 includes opposing first and second ends 22 and 24, opposing third and fourth ends 26 and 28, and opposing first and second sidewall surfaces 30 and 32. Scratch assay device 10 can be disposed on an axis A extending through central body 16 from first end 22 to second end 24 and centrally positioned between third end 26 and fourth end 28. First end includes protrusion 34. Second end can include protrusion 36. First wing 18 includes free end 38. Second wing 20 includes free end 40.

Central body 16 serves as the main platform for the assays and is shaped to fit the dimensions of a standard well 12 in a well plate 14. Central body 16 can be a plate having opposing first and second sidewall surfaces 30 and 32 and a perimeter defined by opposing first and second ends 22 and 24 and opposing third and fourth ends 26 and 28. Well 12 (shown in FIG. 10) is cylindrical. Central body 16 can have a length defined between third and fourth ends 26 and 28 substantially matching an inner diameter of well 12, such that third and fourth ends 26 and 28 can be received in the center of well 12 and engage the inner wall of well 12. This ensures stability and proper alignment of scratch assay device 10 during experimentation.

First and second wings 18 and 20 are located on either side of central body 16 and play a crucial role in holding scratch assay device 10 in place inside well 12. First wing 18 extends outward from first sidewall surface 30 at third end 26. Second wing 20 extends outward from second sidewall surface 32 at fourth end 26. An outer surface of first and second wings 18 and 20 can be flush with third and fourth ends 26 and 28, respectively. First and second wings 18 and 20 extend from central body 16 to free ends 38 and 40, respectively, which are disconnected from central body 16. First and second wings 18 and 20 can have a curved geometry with a curvature substantially matching a curvature of wall of well 12, such that outer surfaces of first and second wings 18 and 20 can engage the wall of well 12. Each of first wing 18 and second wing 20 can form a portion of a semicircle defined between third end 26 and fourth end 28. Free ends 38 and 40 can allow first and second wings 18 and 20 to flex for insertion into well 12.

First and second wings 18 and 20 serve as a compliant mechanism, providing a secure fit and preventing, via friction, any unintended movement or displacement during a cellular growth experiment. This feature is essential for maintaining the accuracy and reliability of the wound healing assays. First and second wings 18 and 20 are biased outward. During insertion into well 12, first and second wings 18 and 20 can be squeezed toward central body 16 to be received in well 20. Upon release, first and second wings 20 engage the inner wall of well 12 providing a friction fit. As discussed further herein, first and second wings 18 and 20 are separated from a bottom of well 12 upon assembly in well 12 such that first and second wings do not interfere with cell growth.

Scratch assay device 10 can be formed of rigid material suitable for engaging the wall of well 12 to provide a secure fit and suitable for cell growth. For example, scratch assay device 10 can formed of a polystyrene or other biocompatible rigid plastic. Any portion of scratch assay device 10 in contact with a bottom of well 12 or cells in well 12 can be formed of a material compatible with cell growth to allow cell growth up to scratch assay device 10. As discussed further herein, scratch assay device can be formed of multiple materials and can include both rigid and compressible materials. Scratch assay device 10 can be formed via 3-dimensional printing technology, injection molding, or other suitable methods of manufacture known in the art.

Scratch assay device 10 can have dual functionality. First end 22 of scratch assay device 10 (shown in FIGS. 1 and 4) is specifically designed for linear scratch assays, where a controlled linear wound is created on a cell monolayer. This enables researchers to study and quantify cell migration and wound closure over time. Second end 24 of scratch assay device 10 (shown in FIGS. 2 and 3) can be designed for circular invasion assays, allowing researchers to investigate cell invasion properties and shifts in polarity through circular wound creation.

First end 22 of central body 16 includes first protrusion 34, which extends outward from first and second wings 18 and 20. First protrusion 34 can include one or more cutouts provided in opposing sidewall surfaces 30 and 32 and extending to a terminal end 42 to reduce a thickness of first protrusion 34 defined between first and second sidewall surfaces 30 and 32. The thickness of first protrusion 34 can be selected based on requirements for cellular migration studies. For example, a thickness of first protrusion 34 measured between first and second sidewall surfaces 30 and 32 can be less than 1 mm or, for example, between 0.25 mm and 0.75 mm. Cutouts can have a uniform depth defined from each of first and second sidewall surfaces 30 and 32 and can extend a partial or full length of first end 22 between third and fourth ends 26 and 28. Terminal end 42 is planar and configured to engage and provide a seal against a bottom of well 12 upon assembly in well plate 14 when scratch assay device 10 is inserted into well 12 with first end 22 down, as described further herein.

Second end 24 of central body 16 includes second protrusion 36. Second protrusion 36 is a cylindrical body configured for circular invasion assays. Second protrusion 36 can be centrally located on second end 24 between third and fourth ends 26 and 28. Second protrusion 36 has terminal end 44 configured to engage and seal against the bottom of well 12 upon assembly in well plate 14 when scratch assay device 10 is inserted into well 12 with second end 24 down.

By offering both linear wound and circular invasion assays in a single tool, scratch assay device 10 provides researchers with flexibility and convenience, eliminating the need for multiple separate devices. Scratch assay device 10 streamlines experimental procedures and reduces the time and effort required for wound healing assays, making it an invaluable asset in cell migration and invasion studies. Overall, scratch assay device 10 offers a practical, user-friendly solution for wound healing assays, combining a well plate-compatible design, secure placement mechanism, and dual functionality for both linear scratch and circular invasion assays.

FIG. 6 shows scratch assay device 10′. Scratch assay device 10′ is substantially similar to scratch assay device 10 with a modified first end 22′. First end 22′ includes first protrusion 34′, which extends outward from central body 16 and first and second wings 18 and 20. First protrusion 34′ is similar to first protrusion 34 of scratch assay device 10 but does not extend fully to third end 26 and fourth end 28. First protrusion 34′ can have a reduced thickness as described with respect to first protrusion 34 and can be centrally located between third end 26 and fourth end 28. First protrusion 34′ has a rectangular shape with terminal end 42′. In one embodiment, terminal end 42′ can be configured to engage and provide a seal against a bottom of well 12 upon assembly in well plate 14 when scratch assay device 10′ is inserted into well 12 with first end 22 down.

To improve the seal against the bottom of well 12, first protrusion 34′ can include shoe 48, shown in FIGS. 7 and 8, which can be formed of a compressible material capable of forming a seal against the bottom of well 12 upon assembly. FIG. 7 shows one example of shoe 48 configured to fit over terminal end 42′ of protrusion 32′. FIG. 8 is a side view of shoe 48. Shoe 48 can include slot 50, which can correspond to a shape of protrusion 34′ such that protrusion 34′ can be received in slot 50. Shoe 48 defines a new terminal end 52 of protrusion 34′ configured to engage and provide a seal against a bottom of well 12 upon assembly in well plate 14 when scratch assay device 10′ is inserted into well 12 with first end 22′ down. Shoe 48 can be formed of a compressible material compatible with cell growth, including, for example, polydimethylsiloxane (PDMS). Shoe 48 can be removably attached to first protrusion 34′.

Shoe 48 can be applied first protrusion 34′ by hand. First protrusion 34′ can be retained in slot 50 of shoe 48 by a press fit. In some embodiments, central body 16 including first protrusion 34′ and second protrusion 36, and first and second wings 18 and 20 can be formed from a rigid material that can be cleaned via autoclaving, while shoe 48 can be removed, disposed of, and replaced in subsequent applications.

As illustrated in FIG. 8, shoe 48 can have walls 52 that extend parallel to walls of slot 50 and walls 54 that angle from walls 52 inward to form terminal end 52. Terminal end 52 can be substantially the same size and shape as terminal end 42 of scratch assay device 10 and described with respect thereto. Terminal end 52 can be compressed against the bottom of well 12 to prevent cells from growing under first protrusion 34′.

In some embodiments, a cylindrical shoe (not shown) can be provided on second protrusion 36 to form a seal against the bottom of well 12 upon assembly.

In some embodiments, first protrusion 32′ of FIG. 6 can, itself, be formed of a compressible and biocompatible material and configured to engage the bottom of well 12 upon assembly. In some embodiments, first protrusion 34″ can be separable from central body 16. FIG. 9 shows a portion of scratch assay device 10″, which is substantially similar to scratch assay device 10′ with modified end 22″ having slot 58 configured to receive a separable first protrusion 34′. Slot 58 can be formed in central body 18 and sized to receive first protrusion 34′ via a friction fit. In this embodiment, first protrusion 34′ can be removed, disposed of, and replaced between applications of scratch assay device 10″.

Slot 58 can have any configuration suitable for receiving and retaining first protrusion 34′. In some embodiments, slot 58 may extend fully across central body 16 opening to one or both third end 26 and fourth end 28 and first protrusion 32′ can be slidably received in slot 58 from one of third end 26 and fourth end 28. In some embodiments, slot 58 and first protrusion 34′ can have corresponding shapes configured to retain first protrusion 34′. For example, first protrusion 34′ can have a neck with bulbous or otherwise shaped larger head configured to be received in a correspondingly shaped slot 58.

In some embodiments, second protrusion 36 can, itself, be formed of a compressible and biocompatible material configured to seal against the bottom wall of well 12 upon assembly. In some embodiments, second protrusion 36 can be, for example, a cylindrical pin configured to be received in a cylindrical slot provided in central body 16 as described with respect to slot 58.

FIG. 10 illustrates one of scratch assay device 10, 10′, and 10″ disposed in well 12 with first end 22, 22′, 22″, configured for linear scratch assays, interfacing with a bottom of well 12, for application of a linear scratch assay. Second end 24, configured for circular invasion assays, faces outward from well 12. For circular invasion assays, scratch assay device 10, 10′, and 10″ can be inserted in the opposite orientation with second end 24 inserted into well 12 to interface with the bottom of well 12.

Scratch assay device 10, 10′, and 10″ is inserted into well 12 of well plate 14 prior to cell culturing. As shown in FIGS. 1, 5, and 6 first protrusion 32, 32′ extends outward of first and second wings 18 and 20 to contact a bottom surface of well 12. First and second wings 18 and 20 are axial displaced from terminal end 42, 42′, 52 and the bottom of well 12 such that first and second wings 18 and 20 do not interfere with cell growth. The portion of scratch device 10, 10′, and 10″ (i.e., terminal ends 42, 42′, 52) contacting the bottom surface of well 12 can be, for example and without limitation, rectangular in shape, as described above. Cells can grow up to the portion of scratch assay device 10, 10′, and 10″ contacting the bottom of well 12, such that when scratch assay device 10, 10′, and 10″ is removed from well 12, a void is left in the shape of the terminal end 42, 42′, 52 disposed on the bottom of well 12.

As shown in FIGS. 2, 5, and 6, second end 24 has a cylindrical second protrusion 36 extending outward from central body 16 and first and second wings 18 and 20 and configured to contact the bottom surface of well 12 when scratch assay device 10 is inserted into well 12. First and second wings 18 and 20 are axial displaced from terminal end 44 and the bottom of well 12 such that first and second wings 18 and 20 do not interfere with cell growth. Again, cells can grow up to and around second protrusion 36, leaving a circular void when scratch assay device 10 is removed from well 12. The surface area of second protrusion 36 can be sized to accommodate a desired microscope orifice.

Scratch assay device 10, 10′, and 10″ allows an exact geometry to be left void of material in a mono layered cellular culture. This area of missing material in the layer of cells is a set shape with an area that is easy to calculate such that when cells grow to enclose the void, tracking their movements is not only easier but more precise than prior art methods.

Scratch assay device 10, 10′, and 10″ can be inserted in well 12 by hand by squeezing first and second wings 18 and 20 toward central body 16 to fit within the inner diameter of well 12. Once first and second wings 18 and 20 are partially received in well 12, scratch assay device 10, 10′, and 10″ can be pressed against the bottom wall of well 12. The position of scratch assay device 10, 10′, and 10″ can be retained in well 12 by an interference fit formed between the wall of well 12 and third and fourth ends 26 and 28 of central body 16 and outer surfaces of first and second wings 18 and 20. Scratch assay device 10, 10′, and 10″ can be sized to allow for removal from well 12 by hand without disruption cell growth in well 12.

Scratch assay device 10, 10′, and 10″ includes several specific technical features that distinguish it from existing technologies in the field of wound healing assays. Some of the notable technical features and their differentiating factors include:
(1) Compatibility with standard well plates: Scratch assay device 10, 10′, and 10″ is specifically designed and scaled to fit directly into standard well plates commonly used in laboratory settings. This compatibility allows for seamless integration into existing experimental workflows, eliminating the need for additional specialized equipment, training, or modifications to experimental protocols. Integration into well cell culturing plates makes scratch assay device 10, 10′, and 10″ efficient and easy to use, simplifying the process and saving time. The user-friendly features minimize the risk of damaging the cell monolayer during scratch creation. Furthermore, the standardized design of scratch assay device 10, 10′, and 10″ ensures consistent scratch size and shape, reducing variability and allowing for accurate comparisons between samples. In contrast, some existing technologies require separate platforms or modifications to the experimental setup, adding complexity and potentially affecting experimental consistency.
(2) Dual functionality: Scratch assay device 10, 10′, and 10″ offers a dual functionality, enabling both linear scratch assays and circular invasion assays. First end 22, 22′ is dedicated to linear scratch assays, facilitating controlled linear wound creation and quantification of cell migration and wound closure. Second end 24 is designed for circular invasion assays, allowing researchers to study cell invasion properties. This 2-in-1 tool provides versatility and efficiency compared to existing technologies that often require separate tools or setups for different types of assays.
(3) Compliance mechanism: The inclusion of first and second wings 18 and 20 in scratch assay device 10, 10′, and 10″ serves as a compliance mechanism that holds scratch assay device 10, 10′, and 10″ securely in place within well 12 of well plate 14. This feature ensures stability during the experiment, minimizing unintended movement or displacement, and maintaining consistent contact with the cell monolayer for improved reproducibility. In contrast, some existing technologies rely on manual or makeshift methods to secure the device, which may lead to variability and inconsistency in the experimental results.

By combining these technical features, scratch assay device 10, 10′, and 10″ offers a comprehensive and user-friendly solution for wound healing assays. Use of scratch assay device 10, 10′, and 10″ streamlines experimental procedures, provides standardization and reproducibility, enhances efficiency, and provides researchers with greater flexibility and versatility in their studies. Scratch assay device 10, 10′, and 10″ addresses the limitations and offers novel advantages over existing technologies in the field of wound healing assays.

The standardized design of scratch assay device 10, 10′, and 10″ ensures consistent scratch size and shape, reducing variability and allowing for accurate comparisons between samples.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

Any relative terms or terms of degree used herein, such as “substantially”, “essentially”, “generally”, “approximately” and the like, should be interpreted in accordance with and subject to any applicable definitions or limits expressly stated herein. In all instances, any relative terms or terms of degree used herein should be interpreted to broadly encompass any relevant disclosed embodiments as well as such ranges or variations as would be understood by a person of ordinary skill in the art in view of the entirety of the present disclosure, such as to encompass ordinary manufacturing tolerance variations, incidental alignment variations, transient alignment or shape variations induced by thermal, rotational or vibrational operational conditions, and the like. Moreover, any relative terms or terms of degree used herein should be interpreted to encompass a range that expressly includes the designated quality, characteristic, parameter or value, without variation, as if no qualifying relative term or term of degree were utilized in the given disclosure or recitation.

1. A device for studying cell migration, wound healing, and/or cell-cell interaction in a cylindrical well of a cell culture well plate, the device comprising:
a central body having opposing first and second sidewall surfaces and opposing first and second ends, wherein the first end is configured to engage a bottom of the well;
a first wing extending outward from the first sidewall surface between the first and second ends; and
a second extending outward from the second sidewall surface between the first and second ends;
wherein each of the first wing and the second wing is biased outward to engage an inner wall of the well.

2. The scratch assay device of claim 1, wherein each of the first wing and the second wing has a curved outer surface configured to engage the inner wall of the well.

3. The scratch assay device of claim 1, wherein the first end comprises a first protrusion, the first protrusion extending outward from the first and second wings.

4. The scratch assay device of claim 3, wherein the first protrusion has a terminal end having a rectangular shape.

5. The scratch assay device of claim 4, wherein the first protrusion comprises a compressible and biocompatible material.

6. The scratch assay device of claim 5, wherein the first protrusion comprises a removable shoe, the removable shoe forming the terminal end and formed of the compressible and biocompatible material.

7. The scratch assay device 5, wherein the first protrusion is received in a slot in the central body and removable therefrom.

8. The scratch assay device of claim 3, wherein the central body further includes oppositely disposed third and fourth ends extending perpendicular to the first and second ends, wherein each of the third end and the fourth end is configured to engage the inner wall of the well.

9. The scratch assay device of claim 8, wherein the first protrusion extends a partial length between the third and fourth ends and wherein the first protrusion is disposed inward of each of the third and fourth ends.

10. The scratch assay device of claim 9, wherein the first protrusion has a thickness less than a thickness defined between the first and second sidewall surfaces.

11. The scratch assay device of claim 8, wherein the first wing extends outward from the first sidewall surface and the third end and the second wing extends outward from the second sidewall surface at the fourth end.

12. The scratch assay device of claim 3, wherein the second end comprises a second protrusion, the second protrusion extending outward from the first and second wings.

13. The scratch assay device of claim 12, wherein the second protrusion is cylindrical having a terminal end with a circular profile.

14. The scratch assay device of claim 13, wherein the second protrusion is centrally located on the second end.

15. The scratch assay device of claim 1, wherein the device has an axis extending through the central body between the first and second ends and wherein each of the first wing and the second wing extends a partial axial length of the central body and wherein each of the first wing and second wing is axially displaced from a terminal end of the first end and each of the first wing and the second wing is axially displaced from a terminal end of the second end.

16. The scratch assay device of claim 15, wherein each of the first end and the second end comprises a biocompatible material.

17. The scratch assay device of claim 16, wherein each of the first end and the second end comprises a compressible material.
